(19) United States
(12) Patent Application Publication
(10) Pub. No.: US 2023/0324417 A1
(43) Pub. Date: Oct. 12, 2023

(54) AUTOMATED PLATFORM TO TREAT TRAUMA-INDUCED COAGULOPATHY WITH PERSONALIZED COAGULATION FACTOR CONCENTRATIONS

(71) Applicants: University of Florida Research Foundation, Inc., Gainesville, FL (US); The Regents of the University of Colorado, Denver, CO (US)

(72) Inventors: Damon Ghetmiri, Gainesville, FL (US); Amor Menezes, Gainesville, FL (US); Mitchell Cohen, Gainesville, FL (US)

(21) Appl. No.: 18/190,623

(22) Filed: Mar. 27, 2023

Related U.S. Application Data
(60) Provisional application No. 63/324,323, filed on Mar. 28, 2022.

(51) Int. Cl. G16H 20/00; G16B 5/00; G01N 33/86; G01N 33/49

(52) U.S. Cl. CPC G01N 33/86; G01N 33/4905; G16B 5/00; G16H 20/00; G01N 2333/974

(57) ABSTRACT

The present disclosure presents systems and methods for administering blood products having a personalized concentration of coagulation factors. One such method includes obtaining measured coagulation factor concentrations from a blood sample of a subject; generating a clotting prediction for the subject based on the measured blood factor concentrations of the subject; determining one or more coagulation factor concentrations to be administered to the subject based on the clotting prediction; iteratively generating a new clotting prediction for the subject based on the determined coagulation factors; iteratively determining additional coagulation factor concentrations to be administered to the subject based on the new clotting prediction until the subject’s coagulation factor concentrations are predicted to equilibrate at a predefined normal range; and/or outputting a recommended set of coagulation factor concentrations to be administered to the subject based on the determined coagulation factor concentrations. Other methods and systems are also provided.

### CROSS-REFERENCE TO RELATED APPLICATION

This application claims priority to co-pending U.S. provisional application entitled, “Automated Platform to Treat Trauma-Induced Coagulopathy with Personalized Coagulation Factor Concentrations,” having serial number 63/324,323, filed Mar. 28, 2022, which is entirely incorporated herein by reference.

### BACKGROUND

There is a dire need for targeted approaches to improve trauma patient treatment outcome. Trauma is the leading cause of death between the ages of 1-44 in the U.S.; those who survive suffer huge morbidity and are left with permanent disabilities. Trauma-induced coagulopathy (TIC) occurs after severe trauma and shock, is biologically characterized by perturbations to the balance between clotting and fibrinolysis, and is clinically characterized by uncontrolled bleeding and either death or clotting complications in those who survive. The initial traumatic hemorrhage accounts for the majority of all trauma-related deaths, and 50% of the mortalities of critically injured patients who undergo surgery. Targeting coagulation biology and the resuscitation strategy in the first 24 hours of care are critical, since 80% of deaths from hemorrhage occur within this window.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1A is a block diagram illustrating an exemplary computing system or device that can be utilized for systems and methods of the present disclosure.

FIG. 1B shows an exemplary coagulation treatment system in accordance with embodiments of the present disclosure.

FIG. 1C shows an implementation of an exemplary coagulation treatment system as a point-of-care device in accordance with embodiments of the present disclosure.

FIG. 2 shows a process of clot formation according to the biochemical kinetics of the coagulation cascade.

FIGS. 3A and 3B show heatmap and bar chart representations illustrating changes in coagulation factor concentrations over time, respectively.

FIG. 4A shows plots of trauma patient coagulation factor concentration time history over the first 24 hours after hospital admission for factors II, V, VII, VIII, IX, X, ATIII, and protein C of 252 trauma patients that converge to equilibrium values within normal ranges.

FIG. 4B shows plots of trauma patient coagulation factor concentration time history over the first 24 hours after hospital admission for factors II, V, VII, VIII, IX, X, ATIII, and protein C that converge to equilibrium values outside of normal ranges of 96 trauma patients that died in the first 24 hours after hospital admission.

FIG. 5A shows a table presenting a sorted correlation of coagulation factor concentrations to model parameters from highest to lowest in accordance with various embodiments of the present disclosure.

FIG. 5B shows plots indicating model improvement for four edge cases of minimum peak, maximum peak, minimum peak-time, and maximum peak-time parameters in accordance with various embodiments of the present disclosure.

FIG. 5C shows tables comparing mean relative error and standard deviation relative error of three Calibrated Automated Thrombogram (CAT) parameters estimated using the old model and the new thrombin dynamics model for 40 trauma patient samples.

FIG. 5D shows a table confirming that model predictions are valid with acceptable mean percent error for five unique divisions (folds) of an original dataset.

FIG. 5E shows plots of CATs predicted with the newly improved thrombin dynamics model in accordance with embodiments of the present disclosure.

FIGS. 6A-6C show plots of pole locations for the newly thrombin dynamics model that demonstrate the effect on system dynamical behavior by individual coagulation factors in accordance with the present disclosure.

FIGS. 7A-7C presents a pseudocode implementation of one embodiment of an exemplary control algorithm in accordance with various embodiments of the patent application.

FIGS. 8A-8C provide a flowchart diagram of one embodiment of an exemplary control algorithm in accordance with various embodiments.

FIG. 9A shows plots of CAT changes from -50% to +50% of initial coagulation factor concentrations in accordance with the present disclosure.

FIG. 9B shows patient-specific mappings from coagulation factor concentration changes to estimated CAT properties in accordance with the present disclosure.

FIG. 10A provides a comparison of CATs over 24 hours by estimating CAT trajectories from the coagulation factor concentrations in accordance with the present disclosure.

FIG. 10B provides a comparison of a recommended patient-specific CAT trajectory compared to an actual CAT trajectory in accordance with the present disclosure.

FIG. 11 shows a comprehensive table of reagents and resources that were used to conduct experiments of the present disclosure.

FIG. 12 shows a plot of poles of a transfer function of the newly improved thrombin dynamics model in the complex plane.

### DETAILED DESCRIPTION

The present disclosure relates to coagulation treatment systems and methods for computing coagulation factor concentrations that rectify clotting in a trauma patient in order to quantitatively guide trauma patient coagulation factor levels while accounting for protein interactions. Exemplary systems and methods utilize an improved thrombin dynamics model to capture the coagulation process to control, use rapidly measurable concentrations to help predict patient state and individual clotting dynamics, and account for patient-specific effects and limitations when adding coagulation factors to remedy coagulopathy by following a novel ordering in which to tune coagulation factors. Validation of an exemplary system/method show superior performance over clinical practice in attaining normal coagulation factor concentrations and normal clotting profiles simultaneously.

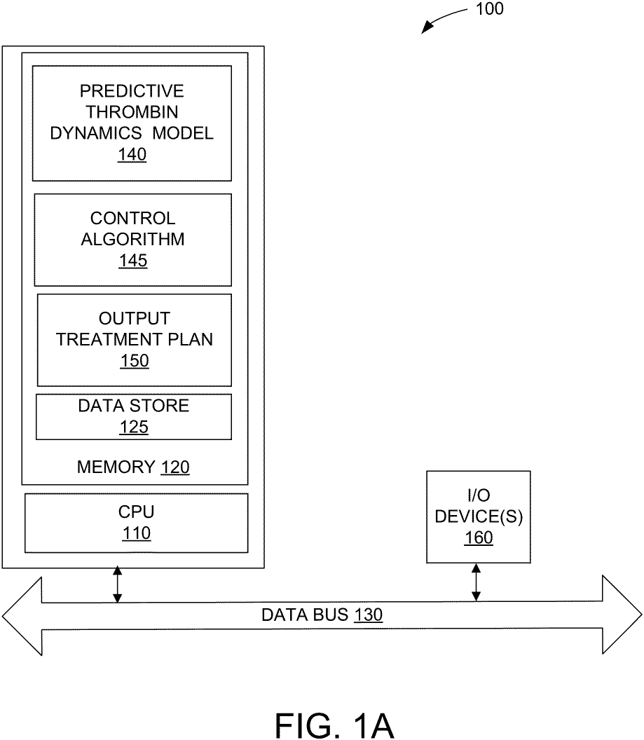

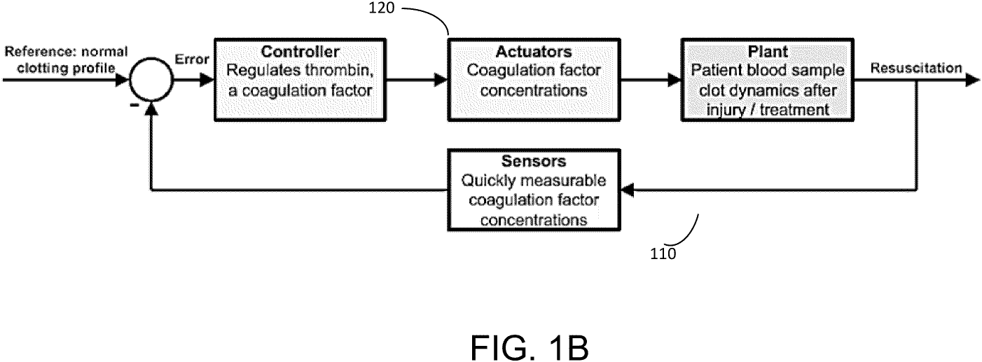

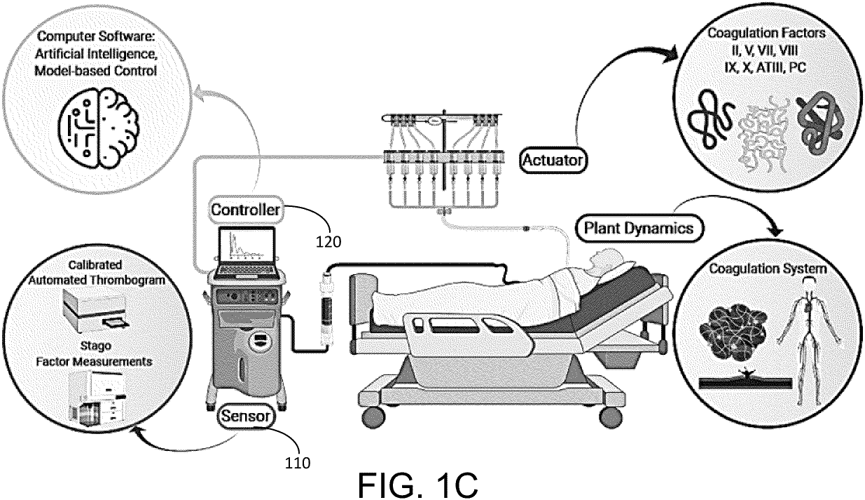

FIG. 1A is a block diagram illustrating an exemplary computing system or device 100 that can be utilized for systems and methods of the present disclosure. Computing system 100 includes at least one processor, e.g., a central processing unit (CPU), 110 coupled to memory elements 120 through a data bus 130 or other suitable circuitry. Computing system 100 stores program code within memory elements 120. Processor 110 executes the program code accessed from memory elements 120 via the data bus 130. In one aspect, computing system 100 may be implemented as a computer or other data processing system. It should be appreciated that computing system 100 can be implemented in the form of any system, such as a controller system, including a processor and memory that is capable of performing the functions described within this disclosure.

Memory elements 120 include one or more physical memory devices such as, for example, a local memory and one or more storage devices. Local memory refers to random access memory (RAM) or other non-persistent memory device(s) generally used during actual execution of the program code. Storage device may be implemented as a hard disk drive (HDD), solid state drive (SSD), or other persistent data storage device. Computing system 100 may also include one or more cache memories (not shown) that provide temporary storage of at least some program code in order to reduce the number of times program code must be retrieved from storage device during execution.

Stored in the memory 120 are both data and several components that are executable by the processor 110. In particular, stored in the memory 120 and executable by the processor 110 are code for a predictive thrombin dynamics model 140, a control algorithm 145 and code for outputting a predictive outcome from the predictive model such as a treatment plan 150. Also stored in the memory 120 may be a data store 125 and other data. The data store 125 can include an electronic repository or database relevant to predictive model results. In addition, an operating system may be stored in the memory 120 and executable by the processor 110. In an embodiment, predictive model data are stored in the data store 125, such as model parameters.

For example, a predictive model may include a digitally constructed model of a probability of individual clotting dynamics. In this context, the model refers to an electronic digitally stored set of executable instructions and data values, associated with one another, which are capable of receiving and responding to a programmatic or other digital call, invocation, or request for resolution based upon specified input values, to yield one or more stored output values that can serve as the basis of computer-implemented recommendations, output data displays, or machine control, among other things. Persons of skill in the field find it convenient to express models using mathematical equations, but that form of expression does not confine the models disclosed herein to abstract concepts; instead, each model herein has a practical application in a computer in the form of stored executable instructions and data that implement the model using the computer. The model may include a model of a current status and/or a model of predicted events of one or more fields (e.g., predicted Calibrated Automated Thrombogram (CAT) trajectories from coagulation factor concentrations). Model and field data may be stored in data structures in memory, rows in a database table, in flat files or spreadsheets, or other forms of stored digital data.

Input/output (I/O) devices 160 such as a keyboard, a display device, and a pointing device may optionally be coupled to computing system 100. The I/O devices may be coupled to computing system 100 either directly or through intervening I/O controllers. A network adapter may also be coupled to computing system to enable computing system to become coupled to other systems, computer systems, remote printers, and/or remote storage devices through intervening private or public networks. Modems, cable modems, Ethernet cards, and wireless transceivers are examples of different types of network adapter that may be used with computing system 100.

Current trauma treatments involve rules-of-thumb and lab-based resuscitation guidelines. In most centers, a preset ratio of blood products is administered to rapidly control hemorrhage. Although some studies attribute improved outcomes to such resuscitation control, other studies show the opposite, including conflicting data for the prehospital transfusion of fresh frozen plasma (FFP) and red blood cells (RBC), and for different ratios of blood products. A possible reason is the dynamic nature of patient coagulation state; too much or too little of beneficial static interventions may result in poor outcomes because of a targeting mismatch with resuscitation needs at that timepoint. While well-intentioned, blood product transfusion is linked to inflammatory morbidities and side-effects including acute respiratory distress syndrome and multi-organ failure. Despite much research and vast improvements in clinical care, severely injured patients that require massive transfusions still have 30% mortality.

Hence, trauma patients may benefit from a tailored transfusion strategy, or from innovative treatments that include coagulation factor (blood protein) concentrates. Targeting individual coagulation proteases via coagulation factor concentrates has benefit for hematologic diseases, such as hemophilia. Although the kitchen-sink approach of using FFP in TIC has its proponents, targeted coagulation factor therapy may have better outcomes compared to FFP-based treatments. However, some reports on modulating coagulation factors (including factor VII, factor IX, and factor X) have shown limited benefit in individually correcting coagulopathic hemorrhaging. Moreover, coagulation factor levels cannot be increased in isolation. For example, elevated levels of activated protein C (aPC) inhibit hemorrhaging, but are also associated with undesirable outcomes including pneumonia, multi-organ failure, and death. Thus, there are open requirements to: (1) confirm the benefits of modulating coagulation factors; and (2) develop a new quantitative modulation approach that incorporates interactions between coagulation factors.

Existing protocols for such goal-driven trauma treatment use thromboelastometry, a viscoelastic coagulation assay, but this assay is time-consuming at typically about an hour per run. These protocols using thromboelastometry are non-quantitative, rely on clinical intuition and older standard procedures, and only correct for a small number of coagulation factors and/or their interactions. Such protocols are qualitative because traditional statistical analysis and machine learning on a static trauma patient measures like coagulation factor concentrations and are not informative in diagnosing coagulation or treatment outcomes. These protocols are also non-dynamic, meaning that they are unable to make time-course, patient-specific predictions of recovery, and they do not facilitate future intervention automation.

Because patient responses to trauma are complex and dynamic, with risks in both hemorrhagic and thrombotic states, dynamical systems approaches are preferable since they offer the ability to intervene at any timepoint, or even at multiple timepoints, in a patient’s coagulopathic trajectory. This capability can reduce a need for urgent hospital interventions to improve physiological outcomes, given that there exist numerous unknown or unquantifiable priors such as patient arrival time to hospital, injury severity, co-morbidities, and patient genetics. Dynamical systems models can capture coagulation kinetics and physiological trauma measures to improve treatment. They can also differ in how much mechanistic coagulation knowledge is harnessed, or how much stoichiometry has to be included. In accordance with various embodiments, an exemplary coagulation treatment system 100 of the present disclosure provides a dynamic, goal-oriented, model-based, rapid trauma patient treatment strategy, as demonstrated by architecture in FIG. 1B, comprising blood coagulation sensors 110, actuators, process dynamics, and a controller 120 that uses sensed measurements of coagulation factor concentrations to actuate clotting dynamics by manipulating these concentrations. In an exemplary implementation, as shown in FIG. 1C, the controller 120 can be part of a point-of-care device that enables clinicians to assess, monitor, and alter trauma patient coagulation status.

Toward achieving this vision, the present disclosure describes how to quantitatively attain appropriate trauma patient treatment goals by combining appropriate actuators, blood coagulation sensors 110, thrombin dynamics models, and a control algorithm into an automated treatment delivery platform that can be physically implemented at the point-of-care. For example, blood samples can be readily obtained from trauma patients. By using blood coagulation sensors 110 and coagulation assays, coagulation factor concentrations in the blood sample can be quickly quantified. A controller algorithm can then recommend a personalized treatment plan according to a goal-oriented approach, moving the patient along a recovery trajectory toward healing. An exemplary control algorithm recommends coagulation factor concentrations for administering blood products, which act as interventions to modulate patient coagulation process dynamics, whereby this intervention approach is repeated frequently and the treatment is adjusted dynamically.

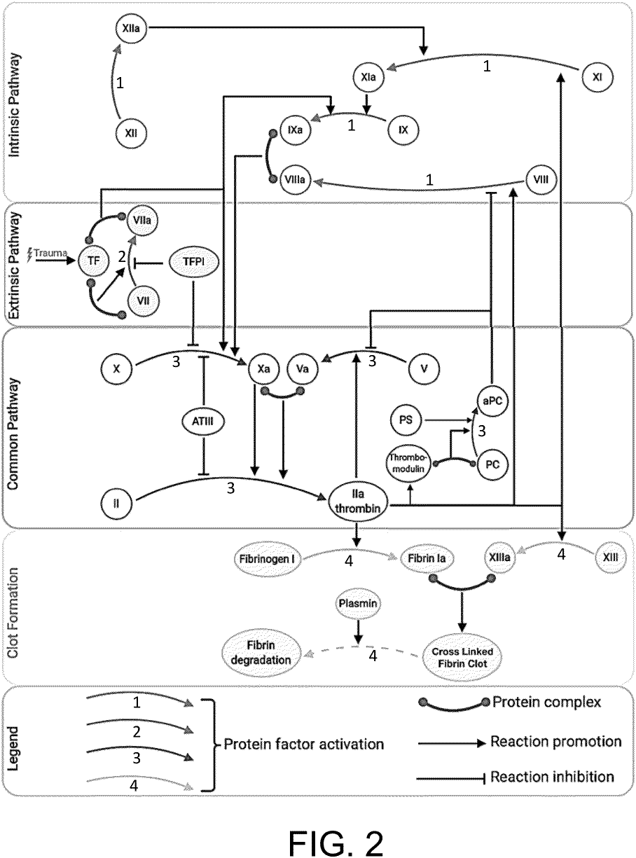

Coagulation factors or proteins are central to an exemplary control approach in that they are the actuators for trauma patient treatment and their concentrations can be rapidly measured within a few minutes using blood coagulation sensors and coagulation assays. The process of clot formation after injury proceeds according to the biochemical kinetics of the coagulation cascade, as shown in FIG. 2, driven by coagulation factor concentrations. Here, patient clotting dynamics, as embodied by the coagulation cascade, consist of biochemical reactions that are initiated following injury. The release of tissue factor (TF) drives the process to generate thrombin, a key end product. Most of the involved proteins, called coagulation factors, are denoted by Roman numerals. An added letter “a” indicates activation. Anticoagulant proteins include tissue factor pathway inhibitor (TFPI), antithrombin (ATIII), protein C (PC), and protein S (PS).

Thrombin, factor IIa, is the end product of the coagulation cascade, and thrombin generation measures can be leveraged to predict hemostatic potential and transfusion requirements. Such measures can replace conventional coagulation tests like prothrombin time (PT), partial thromboplastin time (PTT), international normalized ratio (INR), and platelet counts, all of which have limitations. Thrombin is a unique protein that functions as both a procoagulant and an anticoagulants. As a procoagulant, thrombin activates platelets, converts fibrinogen into strands of fibrin, effects the cross-linking of fibrin to produce a firm fibrin clot by activating factor XIII, and catalyzes other coagulation-related reactions, like the activation of factors V, VIII, XI, and protein C (PC), which in turn regulate thrombin generations. As an anticoagulant, thrombin binds to thrombomodulin, a receptor protein on the endothelial membrane of a blood vessel, initiating a series of reactions that leads to fibrinolysis.

The Calibrated Automated Thrombogram (CAT) is a coagulation assay that can measure the concentration time-history of thrombin in a plasma sample. However, this CAT assay takes about 45-60 minutes to run, without including plasma sample preparation time. Such delays are far too long to be used at a patient’s bedside to predict. and guide treatment and outcomes. An exemplary thrombin dynamics model of the present disclosure can mathematically predict the concentration time-history of thrombin from patient plasma sample coagulation factor concentrations, and that thereby capture the dynamics of the coagulation system process while simultaneously replacing the CAT assay. Such a model is integrated in embodiments of an exemplary controller of a point-of-care device that enables clinicians to assess, monitor, and alter trauma patient coagulation status. In accordance with the present disclosure, a treatment process that leverages such a model can provide frequent, personalized, and dynamic recommendations based on sample clotting predictions to move a trauma patient’s coagulation state toward a desired recovery trajectory.

Conventional trauma patient therapy does not yet use quantitative coagulation factor concentration guidance, possibly because common static machine learning approaches on typical patient data with coagulation factor concentrations are uninformative. Moreover, initial biomarker and injury measurements are not correlated to treatment received, and so cannot predict resuscitation need and adverse outcomes. We found that the means of trauma patient coagulation factor concentrations do not indicate if a trauma patient is at high risk for mortality within 28 days, or at high risk for massive transfusion or a thrombotic event. Equally important, coagulation factor concentrations are uncorrelated to treatment and resuscitation: trauma patients who receive fresh frozen plasma (FFP), no matter the number of units they receive, show substantial variation in coagulation factor concentration changes over time, potentially due to a lack of characterization of, and inherent variability in, coagulation factor concentrations per FFP unit. Therefore, FFP units are not predictive of increases or decreases in coagulation factor concentrations. This also substantiates why FFP administration has mixed results for treatment, since units may not deliver required coagulation factors or may oversupply unnecessary coagulation factors in different patients at different timepoints.

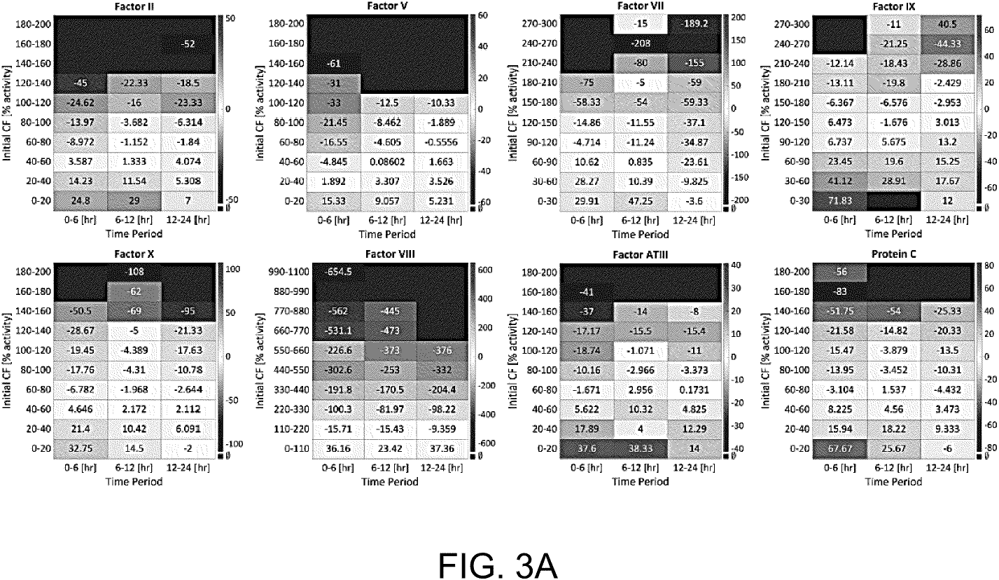

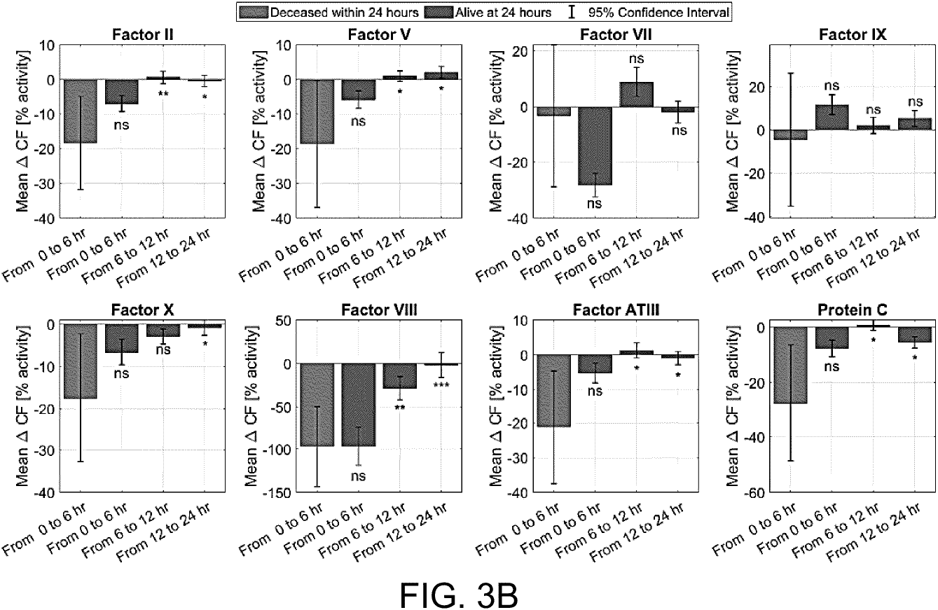

Nevertheless, a close examination of the changes in coagulation factor concentrations for subgroups of 252 survivor trauma patients based on initial coagulation factor levels shows clear dynamic information over the first 24 hours after hospital admission. These dynamics can be illustrated using a heatmap of changes (Δ) in coagulation factor (CF) concentrations at different time periods in the first 24 hours (0h-6h, 6h-12h, 12h-24h), as shown by FIG. 3A. Such changes are computed by subtracting the coagulation factor concentration at the period end time from the coagulation factor concentration at the period start time. For each period, the changes are arranged into heatmap cells according to the coagulation factor concentration at the start of the time period. The mean ΔCF is the number displayed in each heatmap cell, and is matched to an appropriate color.

Specifically, coagulation factor concentrations move toward an equilibrium concentration that is representative of homeostasis, where concentrations that start from a low value increase over time, while concentrations that start from a high value decrease over time, as shown in FIG. 3A. This observation holds true for all coagulation factors. In general, we see darker colors at the lower and upper ends of the FIG. 3A heatmaps at the start time (left side), indicating a sharper change in coagulation factor concentration over the first time period. If the starting concentration of any of factors II, V, VII, VIII, IX, X, ATIII, and protein C is low then the concentration increases, and if the starting concentration is high then the concentration decreases. Numerical values in the cells indicate the mean change of CF in that group, and the cell color represents this mean ΔCF according to the color bar on the right. As coagulation factor concentrations move toward equilibrium over time, the magnitude of these changes decrease, and we observe white and lighter color shades (right side of the heat maps).

To test the significance of the observation that coagulation factor concentrations move toward an equilibrium in patients who recover, a hypothesis (p-value) test was performed that contrasted coagulation factor concentration changes in patients who survived to those who died in the first 24 hours. Four groups were defined as follows: patients who died between 6 and 24 hours, their changes in coagulation factor concentrations between (1) 0 and 6 hours (Deceased 0-6 [hr]); and for patients who were alive at the 24 hour mark post hospital admission time (Alive), their changes in coagulation factor concentrations between (2) 0 and 6 hours, (3) 6 and 12 hours, and (4) 12 and 24 hours. Welch’s t-test was performed and p-values were calculated for α = 0.05. The null hypothesis (H₀) was that the mean change in a coagulation factor’s concentration is equal for patients who are dead or alive, i.e., µₓ= µ_{y} where µₓ and µ_{y} are the deceased and alive sample means, respectively.

FIG. 3B is a bar chart representation of the mean of coagulation factor concentration changes over different time windows, with error bars that indicate a 95% confidence interval. For all coagulation factors, there is no significant difference in concentration changes between the two groups (deceased and alive) from 0 to 6 hours, because the two groups had similar initial conditions. However, in the later time periods in patients who survived, i.e., from 6 to 12 hours and from 12 to 24 hours, there is a significant difference in the mean coagulation factor concentration change of survivors compared to the deceased. The exceptions are for factors VII and IX, due to the large variability of these coagulation factors in the deceased. The results of this analysis reject the null hypothesis and therefore favor an alternative hypothesis Hₐ of non-equal means, i.e., test results indicate that there is enough statistical evidence to conclude that mean changes in coagulation factor concentrations of patients who recovered are significantly different from those of patients who died.

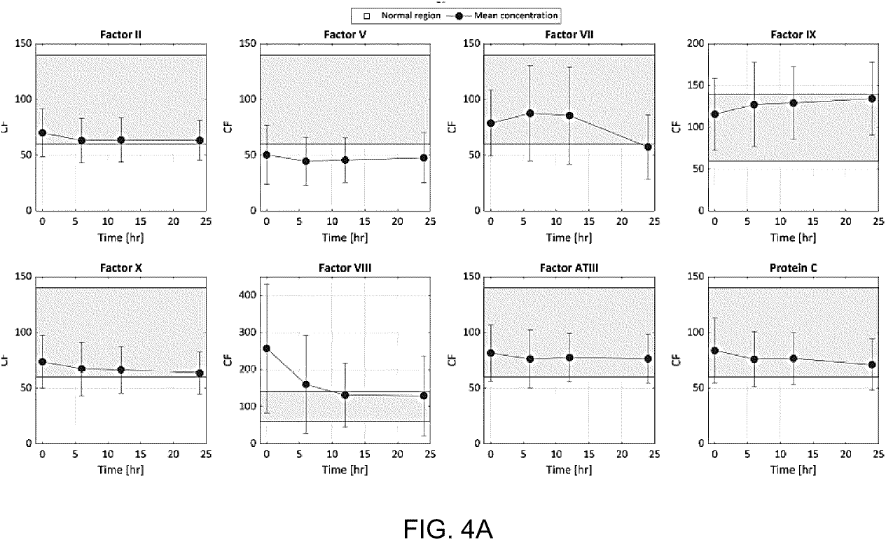

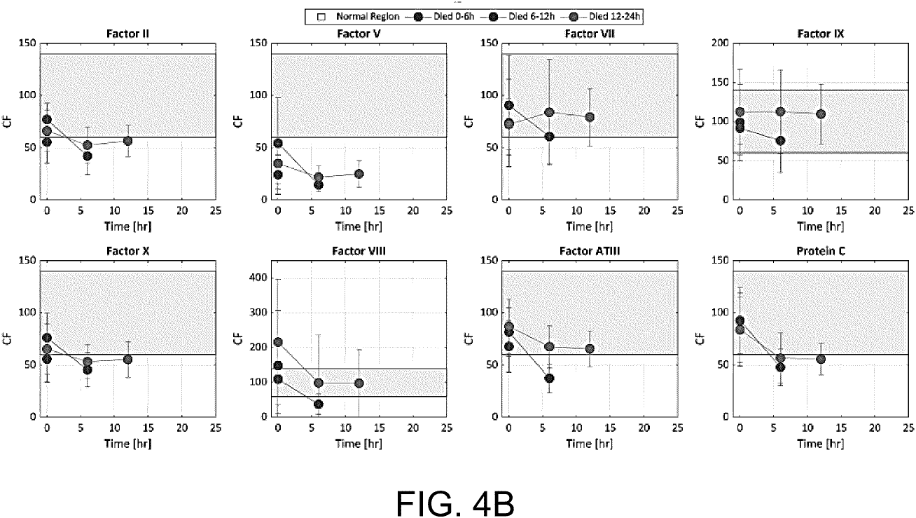

Given that patients who survive the first 24 hours have coagulation factor concentrations that converge to equilibrium values with the change in coagulation factor concentrations moving to zero (either from inadvertent plasma-based modulation of coagulation factor concentrations, or from innate coagulation factor compensation), FIG. 4A shows that these equilibria are within normal ranges of 60-140% activity. Moreover, FIG. 4B shows that trauma patients who die between 6 and 24 hours have coagulation factor concentrations that also converge to equilibrium values, but these are outside normal ranges. It follows that the test data support the claim that a necessary and sufficient condition for trauma patients to survive the first 24 hours is to administer coagulation factors in blood products such that their concentrations will equilibrate at a normal value. The necessary condition is FIG. 4A, and the contrapositive of the sufficient condition is FIG. 4B. Consequently, there is merit to correcting individual coagulation factors dynamically over time, tailored to each patient to improve the treatment outcome.

In order to administer coagulation factors to personalize trauma patient treatment, predictions of the effect of administering coagulation factors are required. Menezes et al. proposed a third-order linear dynamical systems model to rapidly predict CAT trajectories from quickly measured coagulation factor concentrations. While this model has satisfactory prediction capability, the inventors hypothesize that an embedded constraint limits its prediction accuracy and have investigated whether model improvement was possible without changing model structure, by adding a degree-of-freedom parameter to remove this underlying constraint to the input-output model as shown below:

$$\frac{Y \left(s\right)}{U \left(s\right)} = \frac{{K}_{n}}{{s}^{3} + {K}_{2} {s}^{2} + {K}_{1} s + {K}_{0}} {e}^{- {K}_{d} s} , \qquad \text{(1)}$$

where K₀, K₁, K₂, Kₙ, and K_{d} are five patient-specific model parameters (the prior model used four parameters with its fifth parameter constrained), Y(s) is the predicted output thrombin concentration time-history in the frequency domain, and U(s) is a 5 pM impulse input tissue factor (TF) concentration in the frequency domain. An impulse input is an input signal with a very high magnitude that is applied to a system over a very short time. Theoretically, this magnitude approaches infinity as time goes to zero. In practice, this magnitude is taken to be some finite value, commonly 5 pM in the CAT literature, a value that also has experimental justification. Practically, the CAT is instantiated with 5 pM of TF in the plasma sample, which then rapidly depletes.

In accordance various embodiments of with the present disclosure, the initial PC concentration is included with the initial concentrations of factors II, V, VII, VIII, IX, X, and antithrombin (ATIII), creating new linear regressions for the five parameters via the same greedy method, the matching pursuit algorithm. The important role of PC in the coagulation cascade motivated this modification and it has been found that the newly updated thrombin dynamics model substantially improves CAT predictions. Among the few existing thrombin-prediction models, however, important coagulation factors like PC are typically excluded.

For the following discussions, studies performed for the present disclosure were based on normal and trauma patient data arranged into nine datasets (datasets 1, 2, 3, ..., 9). Normal data was obtained from a set of plasma samples from healthy individuals with their CAT and coagulation factor concentration measurements characterized according to standard laboratory protocols. Trauma patient data came from the Activation of Coagulation and Inflammation in Trauma study (ACIT), a previously described single-center prospective cohort study that followed severely injured trauma patients from emergency department admission through discharge from hospitalization or death.

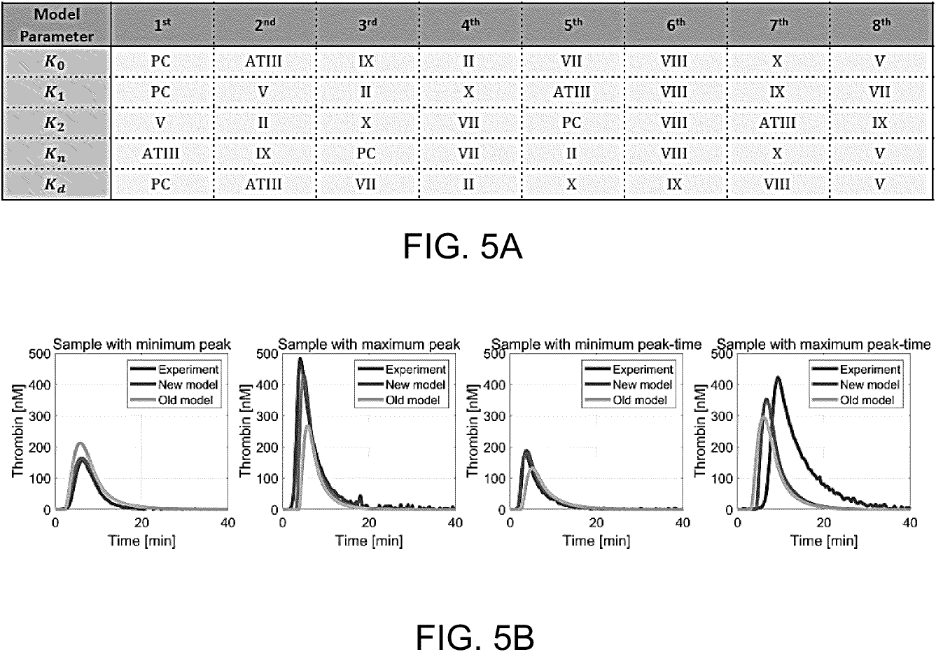

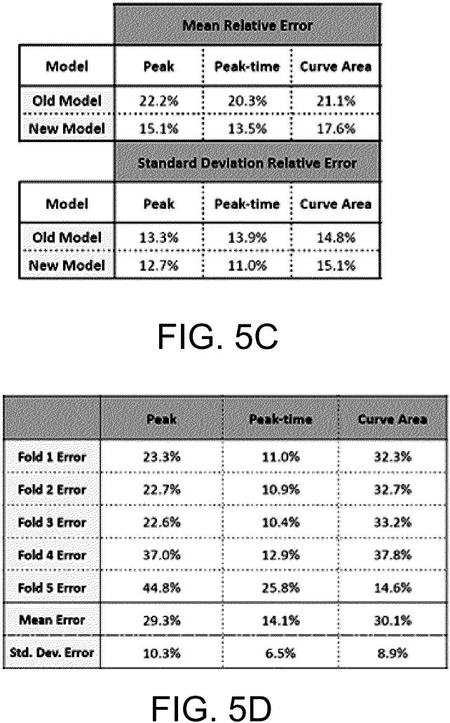

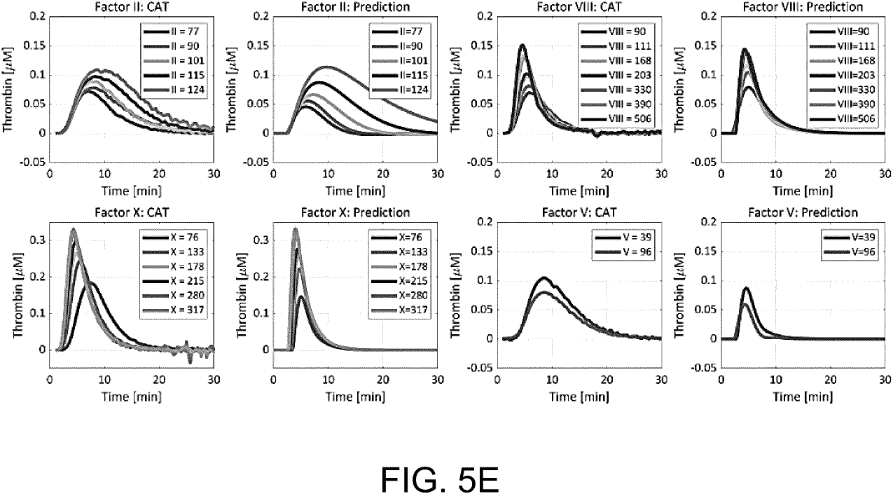

For a dataset of 60 samples (20 individual healthy donors and 40 trauma patients), stepwise linear regression was applied that consists of sequentially and greedily adding the linear effect of a coagulation factor concentration measurement that most reduces the error of a least-squares fit to all data for each of thrombin dynamics model (1) parameters. The coagulation factor that minimizes this least square error has the greatest contribution to the system dynamics captured by that particular model parameter. The stepwise process was repeated until further linear additions of coagulation factor concentration measurements no longer improved the fit. FIG. 5A presents the order of these coagulation factors for each model parameter and confirms the importance of PC and its prime effect on three of the five model parameters.

Visual comparisons of model improvement are shown in FIG. 5B, for four edge cases of minimum peak, maximum peak, minimum peak-time, and maximum peak-time. For trauma patients, the mean peak error improved to 15.1% from 22.2%, the mean peak-time error improved to 13.5% from 20.3%, and the mean thrombin potential (area under the CAT curve) improved to 17.6% from 21.1%, as shown in FIG. 5C. From the figures, FIG. 5A shows that the model fitness improvement of FIGS. 5B and 5C is not because of information increase from adding another coagulation factor to an existing list, but rather because protein C is the most impactful dynamics contributor.

Next, validation of the new improved thrombin dynamics model is presented in two ways: first with five-fold cross-validation and second on a separate dataset that was not used for training. Five-fold cross-validation bootstraps available data by subdividing it so that 80% is used for training and the remaining 20% is used for validation. The process is iterated five times for five unique divisions (folds) of the original dataset. The mean model output properties of these five iterations for the combined dataset of 20 normal samples and 40 trauma patient samples (datasets 4 and 5) are reported in FIG. 5D. This figure confirms good prediction capability. Obtaining errors of 20% or less is a rule-of-thumb for mechanical systems, with less than 10% the ultimate goal through model refinement; given significant inherent biological variability compared to mechanical systems and possible as-yet-undiscovered interactions, a target of 30% or less error is not unreasonable. It is anticipated that model prediction will improve with more trauma CAT data.

Additional model validation was accomplished with a separate validation dataset (dataset 8) that was not used for model training. This validation set started with normal plasma samples that had coagulation factor concentration and CAT measurements, and into which were spiked increasing concentrations of factors II, VIII, and X that were then quantified. An exemplary thrombin dynamics model trained on the separate 60 samples (datasets 4 and 5) can predict the 20 experimental validation CATs (dataset 8) almost perfectly, as shown in FIG. 5E.

To examine the dynamic modulation effects of coagulation factors, three experimental datasets (datasets 4, 5 and 7) were used, where dataset 7 started with normal plasma samples that had coagulation factor concentration and CAT measurements, and into which were spiked increasing concentrations of factors II, VIII, and X. New coagulation factor concentration and CAT measurements were taken after each spike.

The effects of different initial TF concentrations and coagulation factor concentration spikes on system poles were examined. The poles of a dynamical system are characteristic parameters that determine the system’s stability and output response. These poles can be obtained from a transfer function model of a system by determining the values for which the denominator of the transfer function becomes zero, i.e., we find the poles of a trauma patient’s coagulation system by setting the denominator of model (1) to zero and solving the resultant equation for s.

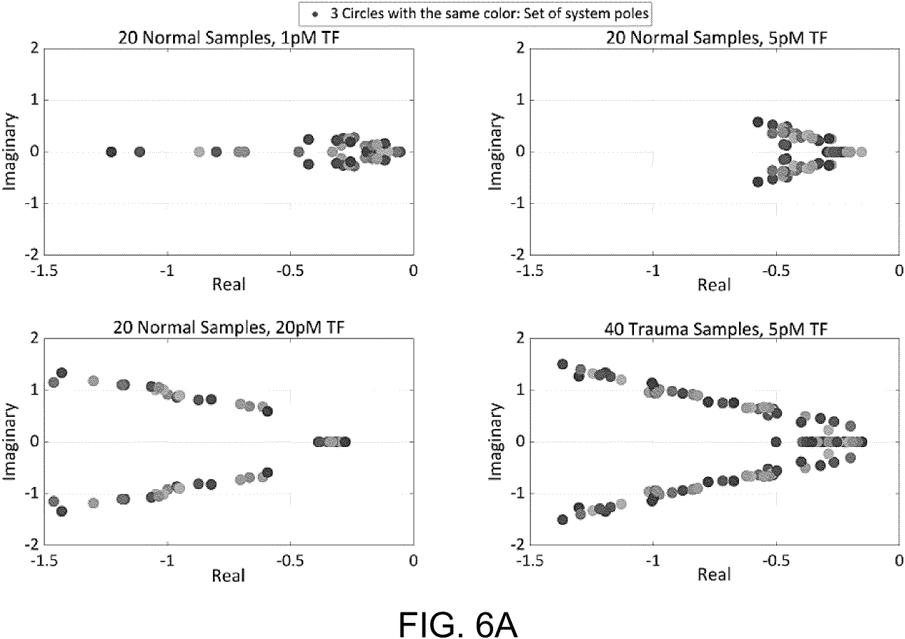

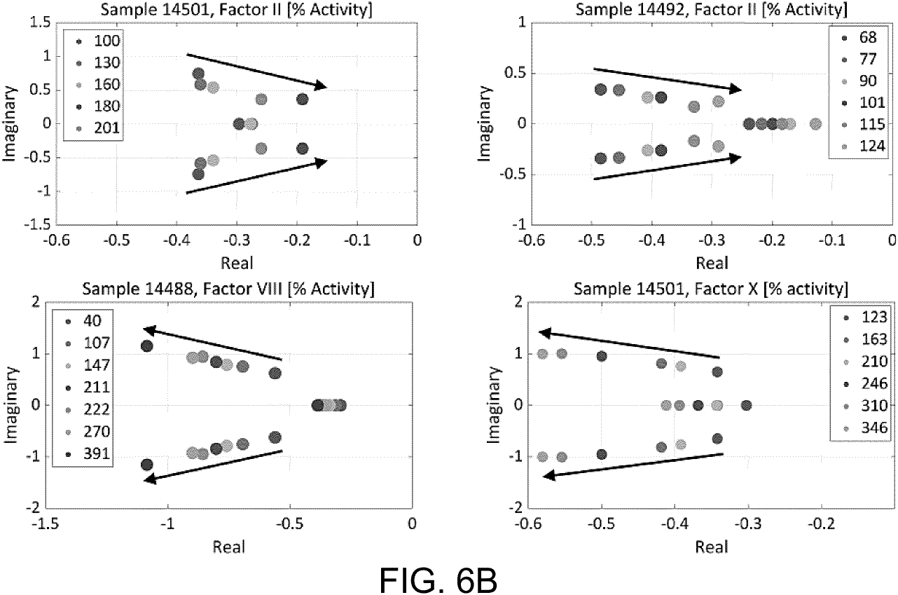

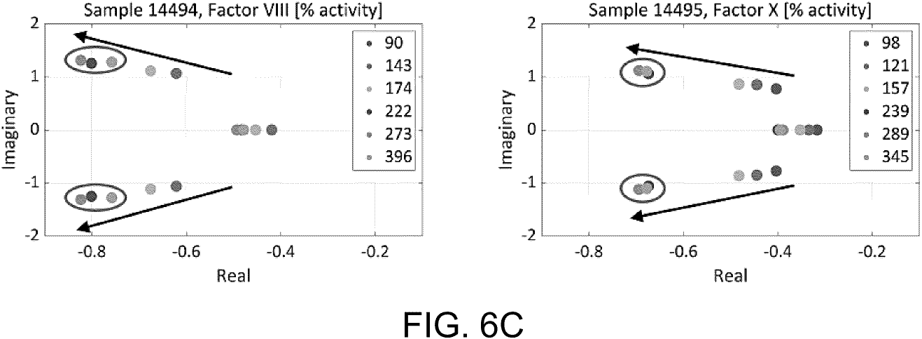

Each coagulation factor has a unique effect on system dynamical behavior as described by the movement of pole locations, and is often accompanied by nonlinear limitations, as illustrated by the figures of FIGS. 6A-6C. The dots in each panel show complex plane pole locations for the transfer function (1) fitted to experimental CATs using the MATLAB Simulink Design Optimization (SDO) toolbox. The three poles of each fit are shown with the same color (in the original figures). FIG. 6A shows pole locations of the fitted transfer functions for 20 normal plasma samples (dataset 4) with inputs of 1 pM TF, 5 pM TF, and 20 pM TF; and 40 trauma patient plasma samples (dataset 5), each with an input 5 pM TF. It can be observed that higher initial TF concentrations move poles away from the origin, and higher initial TF concentrations in normal samples replicate the effects of trauma. FIG. 6B shows that increasing the concentration of factor II in two normal plasma samples moves system poles toward the origin, while increasing the concentration of factors VIII and X in normal plasma samples moves poles away from the origin. FIG. 6C demonstrates that saturation in pole movement is evident for increasing concentrations of factors VIII and X in normal plasma samples. For panels of FIG. 6B and FIG. 6C, numbers in the legend indicate coagulation factor concentration reported as percent activity.

Surprisingly, for 20 normal plasma samples from different donors, we found that increased initial TF concentration caused substantial system pole movement away from the origin, essentially recapturing trauma patient variability, as shown in FIG. 6A. That is, trauma effects are replicable by manipulating TF concentration. Similarly, as FIGS. 6B and 6C show, increases in the concentration of factor II in normal plasma samples pushed coagulation system poles toward the origin, while higher levels of factors VIII and X caused system poles to move away from the origin. Physical limitations like saturation are also apparent in some normal plasma samples, as shown in FIG. 6C, with additional increases in coagulation factor concentrations beyond a certain value not impacting system behavior. It is hypothesized that this observed result is due to the limiting availability of other coagulation factor concentrations that form complexes in the system. The results of spiking isolated coagulation factors into validation plasma samples also validate the actuator effect of each coagulation factor on the human coagulation system and thrombin generation. The isolated increase of each coagulation factor concentration results in a unique change in thrombin profile properties. For example, an increase in factor II leads to an increased peak and increased curve area, an increase in factor VIII mostly only affects the peak value, and an increase in factor X increases peak value and simultaneously reduces peak-time. These effects can be harnessed by an exemplary control algorithm that acts to make a thrombin profile more normal.

To assess personalized control of trauma patient thrombin dynamics using coagulation factors, a target goal CAT and an associated region inside which any CAT trajectories can be considered normal was determined by calculating the maximum, minimum, and mean of the experimental data at each time point for all normal plasma samples, dataset 4 and by fitting model (1) to this data. To evaluate how well the identified region represented normal, the identified region was validated using five normal samples (dataset 9) that were different from dataset 4, which was used for identification. The CAT profile of these five validation samples was contrasted against the normal region using mean relative error (MRE), the mean of the error at each time point where the profile was not within normal minimum and maximum bounds.

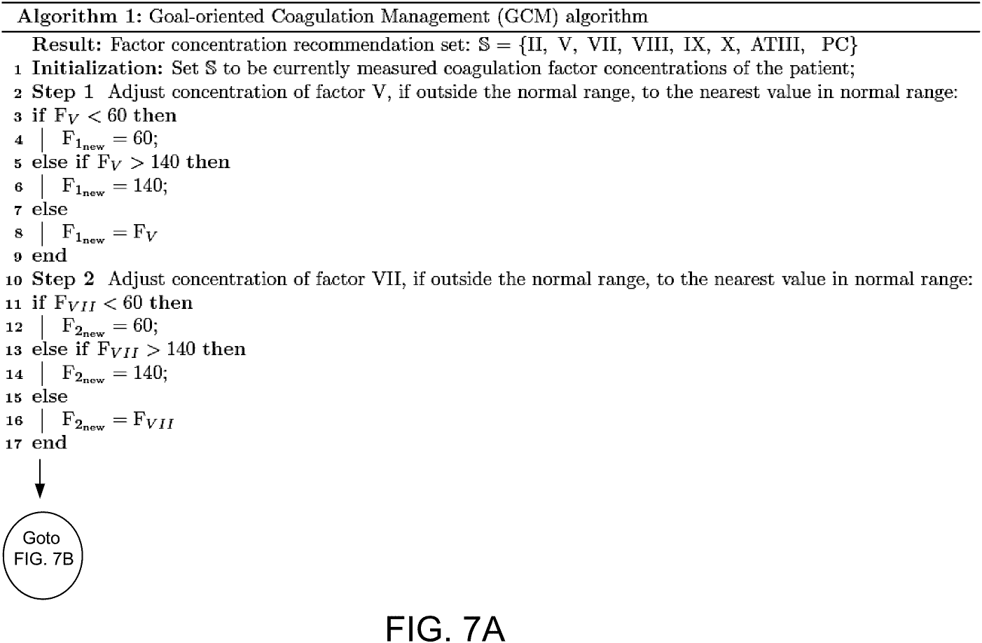

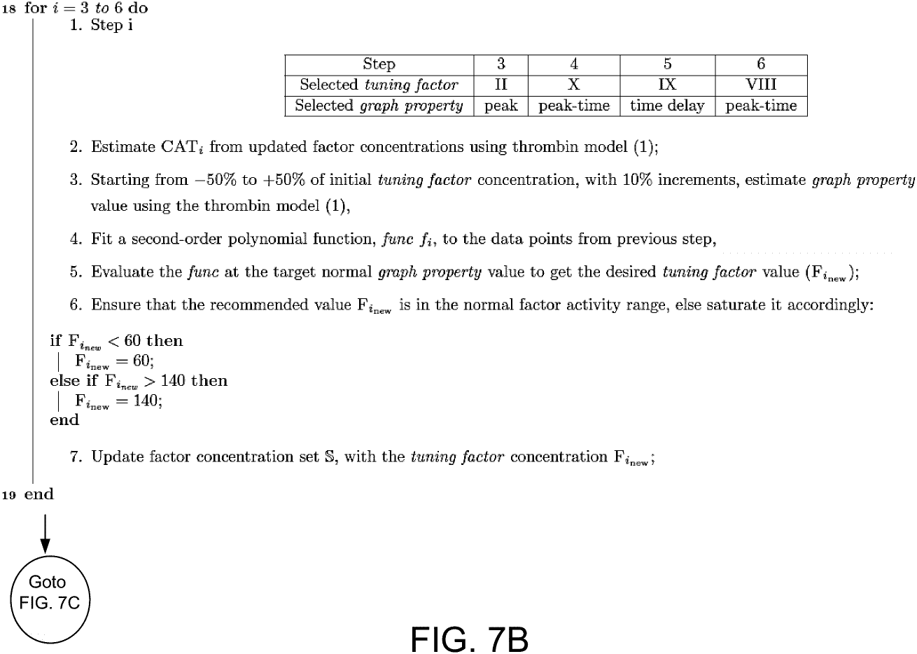

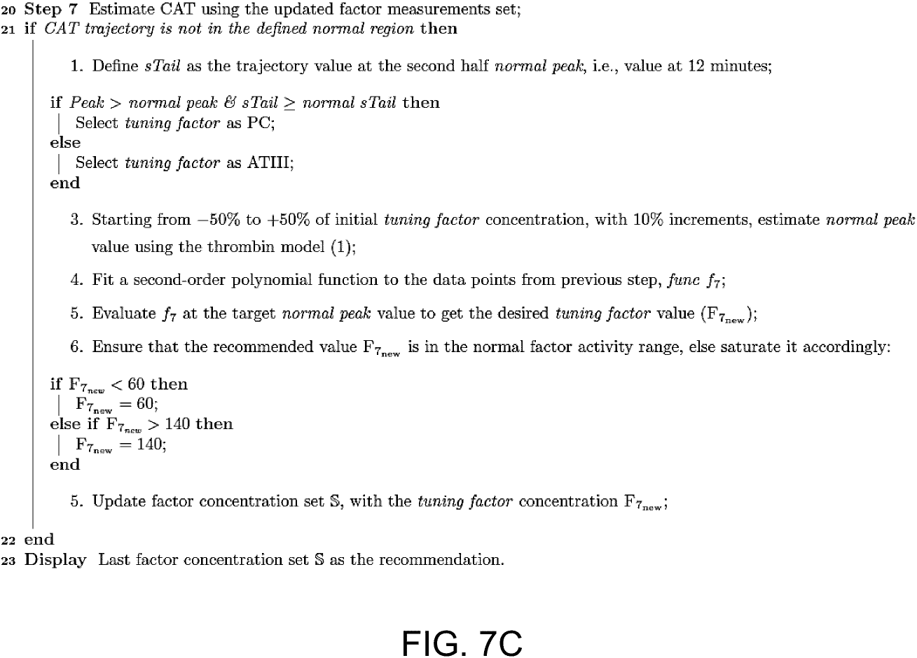

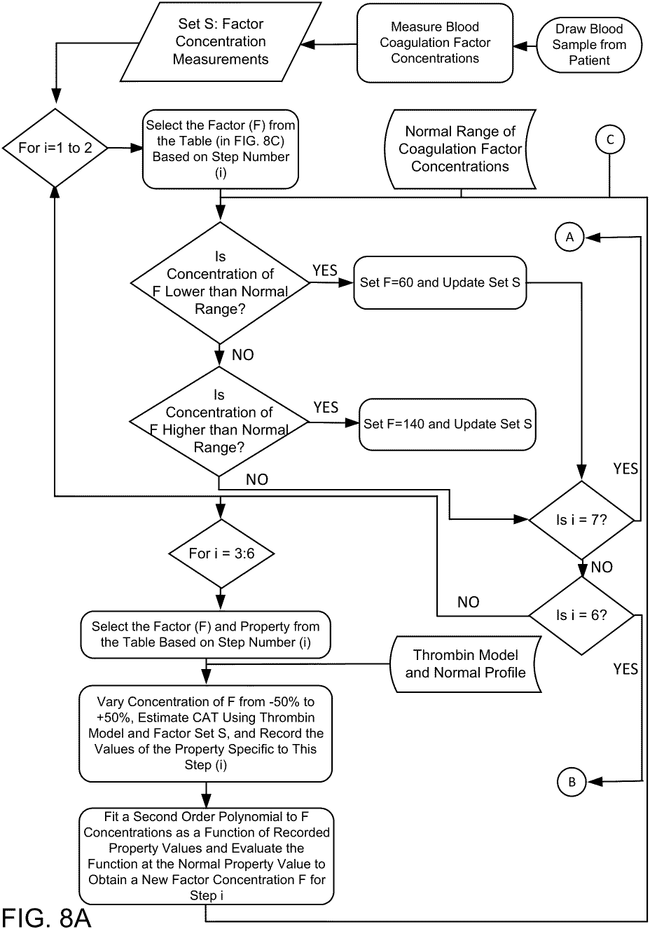

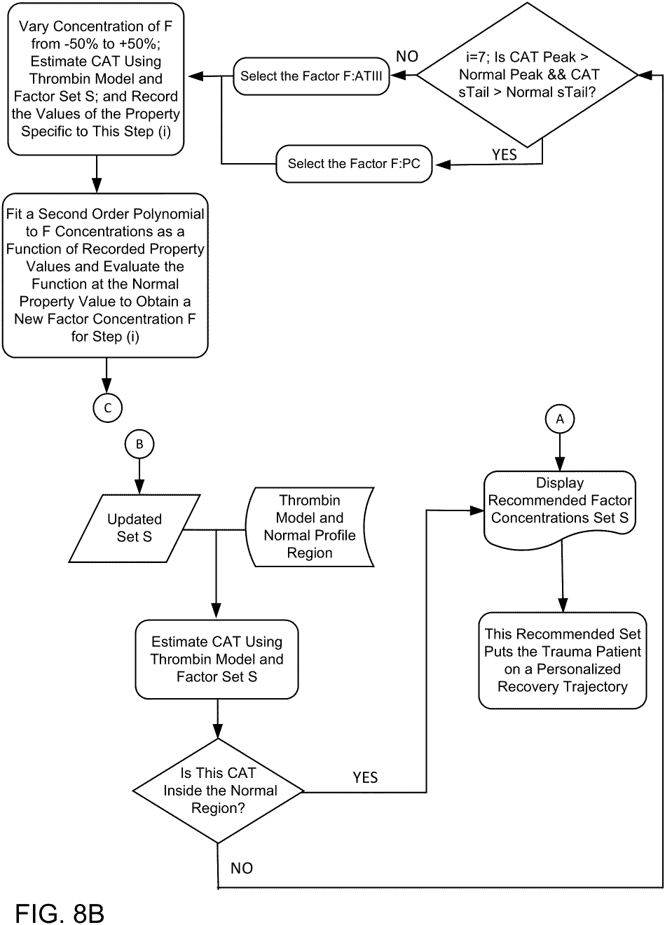

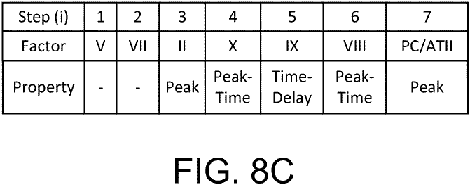

An exemplary control algorithm (also referred to as a “Goal-oriented Coagulation Management (GCM)” algorithm), was developed to recommend a personalized set of coagulation factor concentration changes to move trauma patients onto a recovery path. FIGS. 7A-7C presents a pseudocode implementation of one embodiment of the control algorithm. In turn, FIGS. 8A-8C provide a flowchart diagram of an embodiment of the control algorithm. The exemplary control algorithm enables frequent, dynamic, and personalized TIC treatment. This algorithm systematically recommends coagulation factor concentrations to move a patient CAT trajectory toward normal, while also maintaining concentration values within normal activity ranges. In accordance with various embodiments, the controller 120 of FIG. 1A is configured to operate in accordance with the exemplary algorithm.

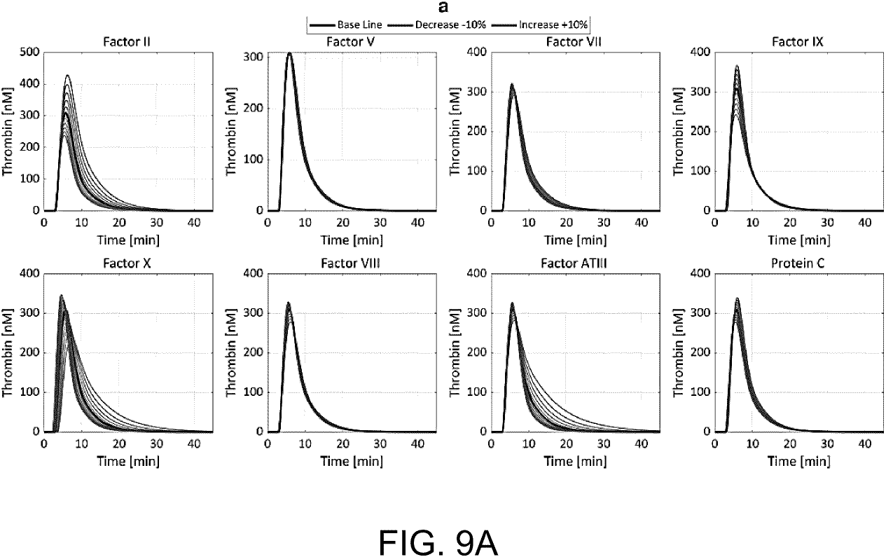

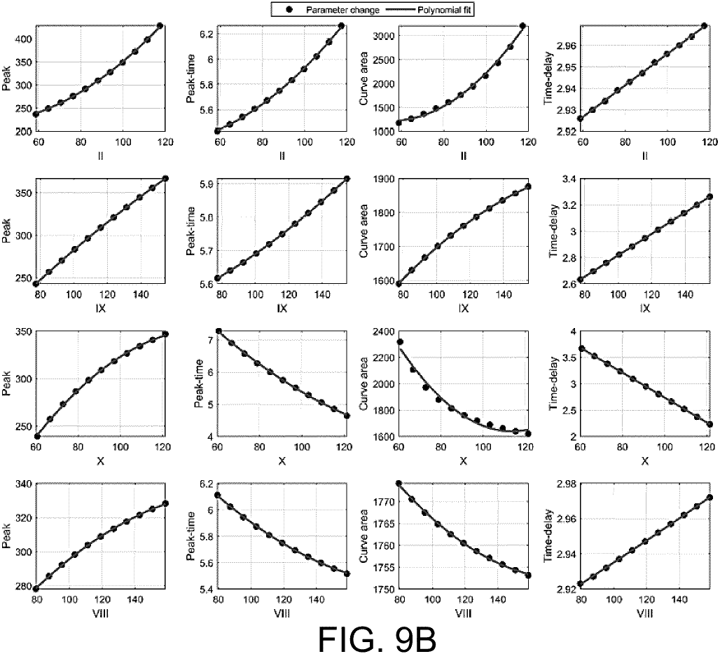

Such an exemplary control algorithm harnesses CAT estimates from coagulation factor concentration measurements via thrombin dynamics model (1), and identifies a patient-specific mapping, as illustrated by FIGS. 9A-9B, from coagulation factor concentration changes to thrombin cloning effects according to these CAT estimates or predictions rapidly and in real-time. In FIG. 9A, CAT changes from -50% to +50% of initial coagulation factor concentrations, in 10% increments, and in FIG. 9B, quadratic polynomial-based patient-specific mappings from coagulation factor concentration changes to estimated CAT properties have excellent fits (mean R² = 0.9996), enabling property manipulation as desired. This mapping is a second-order polynomial, justified by the Akaike Information Criterion as being the smallest-parameter fit that is the most-informative.

Algorithm treatment goals are defined to simultaneously (a) move coagulation factor concentration values toward normal equilibrium values and (b) achieve a normal thrombin (clotting) profile. To attain these treatment goals, the sequence of control algorithm operations was developed by: (i) prioritizing reaching a normal range of coagulation factor concentrations; (ii) ordering how four thrombin profile properties mimic normal clotting; and (iii) investigating the isolated effects of coagulation factors on thrombin profile properties. To satisfy (i), an exemplary control algorithm first corrects concentrations of coagulation factors that have minimal impact on thrombin profile. Thereafter, the predicted CAT is progressively corrected by modulating a coagulation factor concentration according to our new modeled dynamic interactions with the updated concentration checked to be in the normal range at the end of each thrombin profile correction step. For (ii), the control algorithm sets the order in which the algorithm tunes CAT properties as follows: thrombin generation (peak), response time (peak-time), time delay in system response (time delay), and thrombin potential (area under the curve, which is evaluated and compared using the profile tail, called “sTail”). For (iii), the inventors investigated the most impactful individual coagulation factor concentration changes on estimated CAT properties by performing numerical simulations on datasets 4 and 5, as illustrated by FIG. 9, and determined the coagulation factors that have primary and secondary impact on each thrombin profile property. As such, an exemplary control algorithm tunes these coagulation factors to adjust a predicted CAT property in each algorithm step.

Referring to FIGS. 7A-7C and 8A-8C, an exemplary control algorithm first modulates the concentrations of factors V and VII into their normal range because these coagulation factors have limited impact on CAT estimates according to the newly improved thrombin dynamics model, and because their small effects can be overcome by changes in the remaining coagulation factor concentrations as the algorithm progresses. Next, overcoming a trauma patient’s thrombotic or hemorrhagic condition is imperative, equivalent to manipulating a CAT’s peak value. Hence, the algorithm next changes the concentration of thrombin precursor factor II, thereby changing the predicted CAT peak as much as possible while maintaining this coagulation factor’s concentration inside its normal range. Factor X is corrected thereafter, to supplement the peak correction effect of factor II that may be saturated at a normal limit, and also to compensate for changes in peak-time that are caused by factor II manipulation because factor X’s peak-time effect is opposite that of factor II. Factor X also affects the CAT time-delay, which can then be rectified by adjusting the concentration of factor IX with little effect on CAT peak. Modulating factor VIII follows, because changing this coagulation factor allows for fine control of peak-time with minimal effect on CAT peak or time-delay.

The final step of the control algorithm ensures that the recommended CAT estimate is inside the normal region. If not, then the algorithm chooses to manipulate one of two anticoagulant factors, either protein C or ATIII. The choice is made based on a comparison to the area under the normal CAT curve (thrombin potential) in its post-peak stage, based on the differing ways that protein C and ATIII alter the CAT tail. If this normal area is already surpassed by the patient’s updated CAT estimate, then protein C is selected, otherwise protein ATIII is selected. For all of the above modulations, coagulation factors are modulated only to the extent of their predefined normal limits.

For the requisite four “co-” properties that an algorithm is typically scrutinized for, the exemplary control algorithm is convergent, complete, not complex, and correct. First, the control algorithm is guaranteed to converge to a set of personalized coagulation factor concentration recommendations, because the ordered list of a finite number of coagulation factors is systematically manipulated only once through the list. Next, the control algorithm is complete in the sense that if coagulation factor concentration values exist for all eight coagulation factors to generate a simulated CAT trajectory, then the algorithm will output one possible set. Consider that a set of coagulation factor concentrations always exists consisting of the initial coagulation factor concentrations. Indeed, the control algorithm presumes these concentrations at the start before trying to modulate each concentration in turn. Earlier, we showed that the newly improved thrombin dynamics model can accurately predict CAT trajectories from coagulation factor concentrations, those that are measured before algorithm modulation. Thus, completeness is guaranteed. Third, the algorithm’s complexity is linear in the number of coagulation factors n (i.e., it is O(n) in big O notation); there is only one “for” loop in the pseudocode in FIGS. 7A-7C, and the algorithm systematically examines each coagulation factor only once.

Finally, the control algorithm is correct, and its outputs have been validated against clinical outcomes of CAT profile and normalized coagulation factor concentrations for eight trauma patients (dataset 6) who showed methodical recovery toward our normal goal. This eight-patient validation dataset (dataset 6) is different from, and is not a subset of the 40 trauma patients (dataset 5) used for training the thrombin dynamics model (1). An exemplary control algorithm was validated for the first 24 hours post hospital admission as this time period accounts for 80% of hemorrhage fatalities. Intervention periods of 0, 6, 12, and 24 hours were selected for validation and the control algorithm was compared to clinical data because trauma patient data (in dataset 6) were collected at these time points.

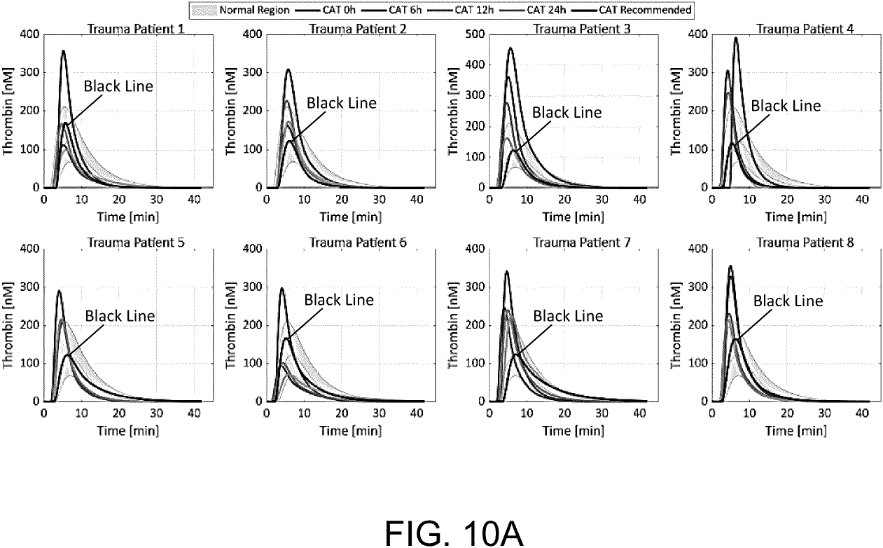

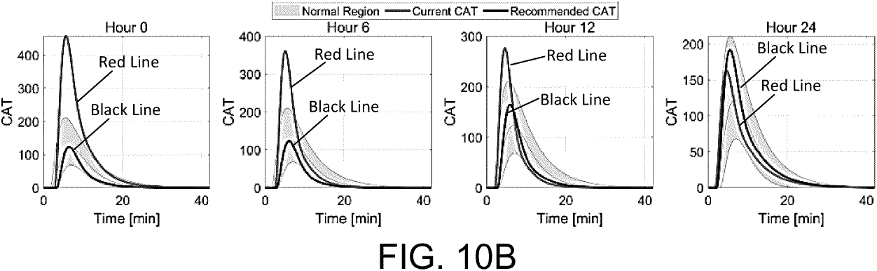

Validation efforts show that control algorithm recommendations drive thrombin generation toward a normal region over time for eight trauma patient samples, validating the correctness and performance of the algorithm. Accordingly, FIG. 10A shows estimated CAT trajectory from coagulation factor measurements for eight trauma patients over 24 hours. The black line shows the control algorithm-recommended patient-specific CAT trajectory at 24 hours if following the personalized coagulation factor recommendations for each patient. All recommended trajectories are visible inside normal ranges. Following the control algorithm recommendations shows desirable improvements over actual treatment received by eight trauma patients in both CAT properties that are quantitatively compared to the normal region criteria and coagulation factor concentrations.

Correspondingly, FIG. 10B illustrates the dynamic performance of the control algorithm over 24 hours for one of these trauma patients. The black line shows the algorithm-recommended patient-specific CAT trajectory compared to the red line representing the actual CAT. This shows how the control algorithm dynamically adjusts the recommendations based on the most recent coagulation factor concentration measurements to move the CAT toward the normal region. In all instances, the recommended CAT is inside the normal region, leading the patient’s thrombin generation toward normal.

Both panels (FIG. 10A and FIG. 10B) show how the control algorithm recommendation adapts according to the most recent coagulation factor concentration measurements to guide the CAT toward the desired normal region. Comparing the recommended goal CAT to the normal region for three CAT properties of peak, peak-time, and area under the curve, the control algorithm’s recommendations show enhanced performance over the clinical practice that occurred, in mean and standard deviation percent error, for all properties over the first 24 hours. The algorithm’s output recommendations also rarely violate the normal CAT region. For the goal of moving coagulation factor concentrations to a normal range, none of the algorithm’s output coagulation factor concentrations violate the normal coagulation factor concentration range, in contrast to 38 violations that occurred during actual treatment of these eight patients at 24 hours, and numerous other violations that occurred at each of several intervening time points.

In summary, the pressing need for trauma patient precision medicine treatments is well-documented, but the coagulopathy problem is complex, which restricts clinicians to using rules-of-thumb, generalized treatment protocols, and uncharacterized blood products. As a result, patient recovery often fluctuates between hypo-coagulable and hyper-coagulable states, with conditions that are complicated by the side effects from contemporary non-tailored approaches. This leads to high mortality and poor outcomes in even the best trauma centers. Goal-oriented, frequent, dynamic, and patient-specific interventions are believed to be the solution, especially if the administration of coagulation factors (blood proteins) can transfer a patient onto a desirable healing trajectory.

Accordingly, exemplary systems and methods of the present disclosure can provide quantitative guidance to assist clinicians at the point-of-care by dynamically adjusting coagulation factors using patient-specifics indicated by an embedded thrombin dynamics model and recommending coagulation factor concentrations that are only within a predefined normal range. The thrombin dynamics model was improved by incorporating an additional parameter to increase model flexibility and by adding the effects of an eighth coagulation factor, protein C, because of its known coagulation importance. These modifications substantially improved the model’s thrombin dynamics predictions, which have been validated on data not used for model training. The present disclosure has verified in silico that administering coagulation factor concentrations changed the clotting that was described by the newly improved thrombin dynamics model. Coagulation factor levels have been chosen to comply with generally accepted normal limits when modulated in a treatment scheme due to observed saturating behavior for excessive coagulation factor concentration administration.

Algorithm prediction performance has been validated in silico on data not used for training by contrasting against metrics from actual trauma patients who recovered and also progressed toward normal. Validation efforts show that an exemplary control method not only guides clotting predictions closer to normal, but does so while maintaining all coagulation factor concentrations within normal ranges, which has not been the case in conventional practice.

The present disclosure offers a personalized control approach to trauma patient treatment by utilizing clotting system dynamics, characterizing the effects and limitations of coagulation factor actuators, and then articulating a control algorithm to systematically achieve coagulation goals in precision trauma patient resuscitation. Such methods and systems can be utilized in physical treatment devices at the point-of-care and can facilitate the automation of frequent, tailored clinical interventions in near real-time. An iterative approach of the present disclosure permits quicker model updates, greater personalization, and a responsiveness to uncertainties, all of which will improve patient outcomes and aid in precision trauma patient resuscitation.

In various embodiments, algorithm-recommended coagulation factor concentration increases in blood samples may be achieved by accurately adding specific recombinant coagulation factors, and algorithm-recommended coagulation factor concentration decreases may be achieved by accurately diluting samples or by augmenting inhibiting coagulation factors. In addition to trauma treatments, the results of the disclosed systems and methods may also be for various coagulation disorders, such as hemophilia, von Willebrand disease, factor V Leiden, pulmonary embolism, deep vein thrombosis, stroke, and sickle cell disease.

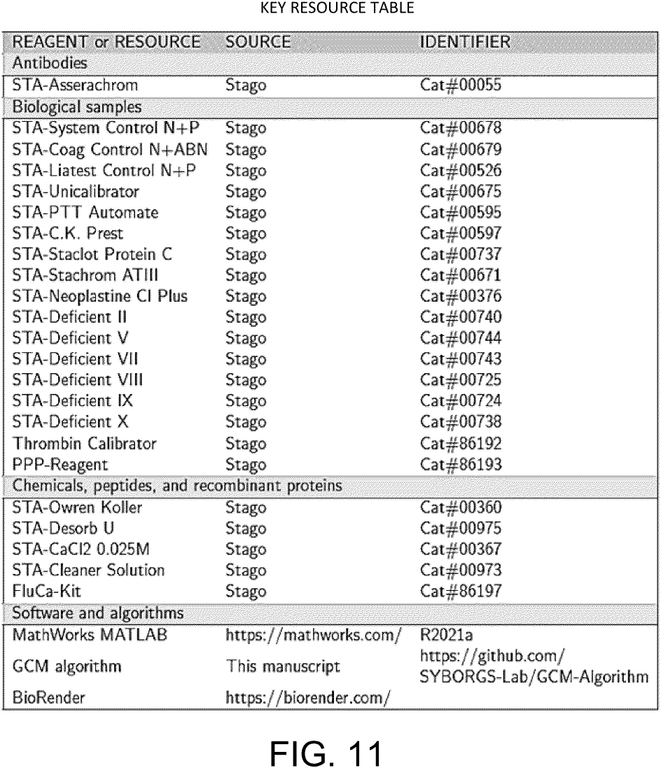

A comprehensive table of all the reagents and resources that were used to conduct experiments, including for coagulation factor measurements and Calibrated Automated Thrombograms, are presented in the Key Resource Table of FIG. 11, along with the electronic resources that were used for simulations and analysis.

Coagulation factor concentrations were measured using the STA Compact Max® benchtop coagulation analyzer (having blood coagulation sensors 110) as percent activity, which is with respect to the normal coagulation factor concentration in a healthy person. A normal range for coagulation factor concentrations is typically 60-140% activity. Plasma samples were removed from -80° C. storage and thawed at room temperature. Reagents were prepared with DiH20 and left to stabilize for 30-60 minutest. Owren-Koller diluent was used for patient samples, STA-Unicalibrator reagent was used to calibrate the system by measuring/defining ranges of new reagent lots (performed monthly), STA-System Control N+P and STA-Coag Control N+ABN were control reagents measured every four hours and eight hours, respectively, and STA-Deficient reagent was used to measure the activity of a coagulation factor, e.g., STA-Deficient V was used for measuring factor V. The test automatically started after loading sample and reagents into the instrument. Given that quality control was repeated every four hours, coagulation factor concentration measurements were performed once for each sample.

For certain studies, plasma sample thrombin expression experimental data was obtained using the ThermoFisher Fluoroskan Microplate Fluorometer with Calibrated Automated Thrombogram software as follows. Plasma samples were removed from -80° C. storage and thawed at room temperature. Reagents were added to 96-well plates: thrombin calibrator reagent was used for the measurement control, and PPP-reagent was used to measure thrombin in normal or trauma samples. Plasma samples were added to plate wells, with three biological replicates, and the plate was loaded into a Fluoroskan Ascent platereader. Following a ten-minute incubation period at 37° C., the test started automatically when the machine dispensed the FluCa reagent, which was pre-loaded. Ultimately, the generated thrombin generation was measured and recorded every 20 seconds. These measurements included three technical replicates.

Correspondingly, model parameters of the newly improved thrombin dynamics model (1) were fit to experimental data using the MATLAB Simulink Design Optimization (SDO) toolbox. The input was defined as an impulse input with the desired magnitude, e.g., 5 pM of TF. The output to fit was the individual CAT profile experimental data. Solver tolerance was set to 1e-9. Starting from an initial parameter guess, the MATLAB SDO toolbox optimized parameter values of a transfer function model by minimizing the least square error between prediction and actual data using a trust region reflective algorithm. Following convergence, the finalized transfer function model parameters for each experimental sample were recorded and the poles computed.

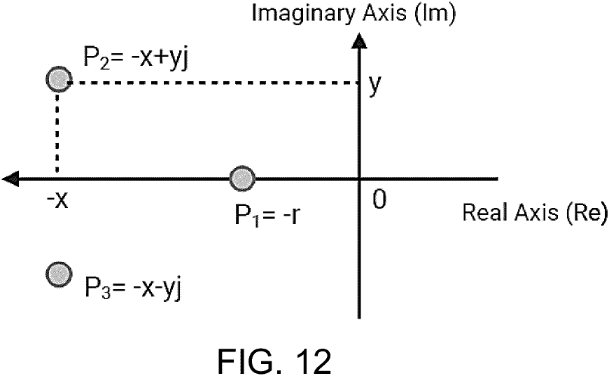

Poles of a transfer function are the values for which the value of the denominator of the transfer function becomes zero. Therefore, to obtain the pole location values, the denominator of the fitted model was set equal to zero and the resultant equation was solved, i.e., solving s³ + K₂s² + K₁s +K₀ = 0. Since this is a third order system, the solution is a set of three numbers with real and imaginary parts, which can be plotted in the complex plane as shown in FIG. 12.

For statistical analysis, the Welch’s t-test was performed for two unpaired samples (deceased, x, versus any one of the alive groups described in the main text, y) using the following equation:

$$t = \frac{{μ}_{x} - {μ}_{y}}{\sqrt{\frac{{S}_{x}^{2}}{n} - \frac{{S}_{y}^{2}}{m}}} ,$$

where µₓ and µ_{y} are the deceased and alive sample means, respectively; Sₓ and S_{y} are the sample standard deviations; and n and m are the sample sizes of x and y, respectively. Next, p-values were calculated for α = 0.05, i.e., 95% confidence interval, using MATLAB’s ttest2 function. The results were reported by ns: not significant, p > 0.05; ∗ : p ≤ 0.05; ∗∗ : p ≤ 0.01; and ∗∗∗ : p ≤ 0.001.

Computer program code for carrying out operations of the present disclosure may be written in a variety of computer programming languages. The program code may be executed entirely on at least one computing device (or processor), as a stand-alone software package, or it may be executed partly on one computing device and partly on a remote computer. In the latter scenario, the remote computer may be connected directly to the one computing device via a LAN or a WAN (for example, Intranet), or the connection may be made indirectly through an external computer.

It will be understood that each block of the flowchart illustrations and block diagrams and combinations of those blocks can be implemented by computer program instructions and/or means. These computer program instructions may be provided to a processor of a general purpose computer, special purpose computer, application specific integrated circuit (ASIC), or other programmable data processing apparatus to produce a machine, such that the instructions, which execute via the processor of the computer or other programmable data processing apparatus, create means for implementing the functions specified in the flowcharts or block diagrams.

It should be emphasized that the above-described embodiments of the present disclosure are merely possible examples of implementations, merely set forth for a clear understanding of the principles of the disclosure. Many variations and modifications may be made to the above-described embodiment(s) without departing substantially from the principles of the disclosure. All such modifications and variations are intended to be included herein within the scope of this disclosure and protected by the following claims.

1. A method for administering blood products having a personalized concentration of coagulation factors comprising:
obtaining, by a computing device, measured coagulation factor concentrations from a blood sample of a subject;
generating, by the computing device, a clotting prediction for the subject based on the measured blood factor concentrations of the subject;
determining, by the computing device, one or more coagulation factor concentrations to be administered to the subject based on the clotting prediction;
iteratively generating, by the computing device, a new clotting prediction for the subject based on the determined coagulation factors;
iteratively determining, by the computing device, additional coagulation factor concentrations to be administered to the subject based on the new clotting prediction until the subject’s coagulation factor concentrations are predicted to equilibrate at a predefined normal range; and
outputting, by the computing device, a recommended set of coagulation factor concentrations to be administered to the subject based on the determined coagulation factor concentrations.

2. The method of claim 1, wherein the clotting prediction comprises a predicted Calibrated Automated Thrombogram (CAT) trajectory from the measured coagulation factor concentrations.

3. The method of claim 1, wherein the clotting prediction is based on a third-order linear dynamics system model having five unconstrained parameters.

4. The method of claim 1, further comprising:
inputting, by the computing device, the measured blood factor concentrations of the blood sample of the subject into a predictive thrombin dynamics model;
executing, by the computing device, the predictive thrombin dynamics model; and
predicting, by the computing device using the predictive thrombin dynamics model, a Calibrated Automated Thrombogram (CAT) trajectory for the subject.

5. The method of claim 4, wherein the measured blood factor concentrations that are input in the predictive thrombin dynamics model comprise initial concentrations of protein C and factors II, V, VII, VIII, IX, X, and antithrombin (ATIII).

6. The method of claim 1, wherein the measured blood factor concentrations are obtained from a blood coagulation sensor that measures blood factor concentrations of the blood sample.

7. The method of claim 1, wherein the recommended set of coagulation factor concentrations move the coagulation factor concentration values of the subject toward normal equilibrium values of the subject.

8. A system comprising:
a processor of a computing device;
a memory in communication with the processor, the memory storing program instructions, the processor operative with the program instructions to perform the operations of:
obtaining, by a computing device, measured coagulation factor concentrations from a blood sample of a subject;
generating, by the computing device, a clotting prediction for the subject based on the measured blood factor concentrations of the subject;
determining, by the computing device, one or more coagulation factor concentrations to be administered to the subject based on the clotting prediction;
iteratively generating, by the computing device, a new clotting prediction for the subject based on the determined coagulation factors;
iteratively determining, by the computing device, additional coagulation factor concentrations to be administered to the subject based on the new clotting prediction until the subject’s coagulation factor concentrations are predicted to equilibrate at a predefined normal range; and
outputting, by the computing device, a recommended set of coagulation factor concentrations to be administered to the subject based on the determined coagulation factor concentrations.

9. The system of claim 8, wherein the clotting prediction comprises a predicted Calibrated Automated Thrombogram (CAT) trajectory from the measured coagulation factor concentrations.

10. The system of claim 8, wherein the clotting prediction is based on a third-order linear dynamics system model having five unconstrained parameters.

11. The system of claim 8, wherein the operations further comprise:
inputting, by the computing device, the measured blood factor concentrations of the blood sample of the subject into a predictive thrombin dynamics model;
executing, by the computing device, the predictive thrombin dynamics model; and
predicting, by the computing device using the predictive thrombin dynamics model, a Calibrated Automated Thrombogram (CAT) trajectory for the subject.

12. The system of claim 11, wherein the measured blood factor concentrations that are input in the predictive thrombin dynamics model comprise initial concentrations of protein C and factors II, V, VII, VIII, IX, X, and antithrombin (ATIII).

13. The system of claim 8, wherein the measured blood factor concentrations are obtained from a blood coagulation sensor that measures blood factor concentrations of the blood sample.

14. The system of claim 8, wherein the recommended set of coagulation factor concentrations move the coagulation factor concentration values of the subject toward normal equilibrium values of the subject.

15. A non-transitory computer-readable medium comprising program instructions that, when executed by at least one computing device, direct the at least one computing device to:
obtain measured coagulation factor concentrations from a blood sample of a subject;
generate a clotting prediction for the subject based on the measured blood factor concentrations of the subject;
determine one or more coagulation factor concentrations to be administered to the subject based on the clotting prediction;
iteratively generate a new clotting prediction for the subject based on the determined coagulation factors;
iteratively determine additional coagulation factor concentrations to be administered to the subject based on the new clotting prediction until the subject’s coagulation factor concentrations are predicted to equilibrate at a predefined normal range; and
output a recommended set of coagulation factor concentrations to be administered to the subject based on the determined coagulation factor concentrations.

16. The non-transitory computer-readable medium of claim 15, wherein the clotting prediction comprises a predicted Calibrated Automated Thrombogram (CAT) trajectory from the measured coagulation factor concentrations.

17. The non-transitory computer-readable medium of claim 15, wherein the clotting prediction is based on a third-order linear dynamics system model having five unconstrained parameters.

18. The non-transitory computer-readable medium of claim 15, wherein the at least one computing device is further directed to:
input the measured blood factor concentrations of the blood sample of the subject into a predictive thrombin dynamics model;
execute the predictive thrombin dynamics model; and
predict, using the predictive thrombin dynamics model, a Calibrated Automated Thrombogram (CAT) trajectory for the subject.

19. The non-transitory computer-readable medium of claim 18, wherein the measured blood factor concentrations that are input in the predictive thrombin dynamics model comprise initial concentrations of protein C and factors II, V, VII, VIII, IX, X, and antithrombin (ATIII).

20. The non-transitory computer-readable medium of claim 15, wherein the measured blood factor concentrations are obtained from a blood coagulation sensor that measures blood factor concentrations of the blood sample.
